(19) United States
(12) Patent Application Publication
(10) Pub. No.: US 2022/0164711 A1
(43) Pub. Date: May 26, 2022

(54) COMPUTERIZED SYSTEM AND METHOD FOR ANTIGEN-INDEPENDENT DE NOVO PREDICTION OF CANCER-ASSOCIATED TCR REPERTOIRE

(71) Applicant: Board of Regents of the University of Texas System, Austin, TX (US)

(72) Inventor: Bo Li, Irving, TX (US)

(21) Appl. No.: 17/440,993

(22) PCT Filed: Mar. 16, 2020
(86) PCT No.: PCT/US2020/022925

§ 371 Date: Sep. 20, 2021

Related U.S. Application Data
(60) Provisional application No. 62/825,235, filed on Mar. 28, 2019.

(51) Int. Cl. G06N 20/20; G06N 3/12

(52) U.S. Cl. CPC G06N 20/20; G06N 3/123

(57) ABSTRACT

Disclosed are systems and methods for a pan-cancer early detection tool that is able to augment the small signals emitted from early and/or late-stage cancer by analyzing and understanding the changes in the blood T cell receptor (TCR) repertoire. The disclosed systems and methods embody an immune-based cancer detection technology that can detect cancer signals from the signatures of the peripheral immune repertoire, which can be performed with high accuracy even at the early stages of the disease. An improved framework is employed that is embodied through a novel machine learning algorithm that can predict cancer status based on a patient's peripheral blood TCR repertoire, such that a deep TCR sequencing of the genomic DNA of the white blood cells is performed, which enables the detection (prediction or determination) of cancer-associated TCRs independent of tumor antigens. This provides a robust biomarker for both early and late-stage cancers across diverse diseases.

### CROSS-REFERENCE TO RELATED APPLICATIONS

This application claims benefit of priority from U.S. Provisional Patent Application No. 62/825,235, filed on Mar. 28, 2019, which is incorporated by reference in its entirety.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### GOVERNMENT INTEREST

There is no government interest or support for this work.

### FIELD

The present disclosure generally relates to an immune-repertoire based cancer diagnosis technology, and more particularly to a novel system and method for diagnosing a patient with cancer and determining his/her cancer status with peripheral blood T cell receptor (TCR) repertoire.

### BACKGROUND

Clinical utilities of immune repertoire sequencing data for cancer diagnosis and prognosis have not yet been fully explored. Current technologies broadly focus on detecting large thresholds of cancer-related materials in the human body. For example, traditional methods for cancer detection rely on identification of cancer biomarkers (e.g., CA antigens in the serum), circulating deoxyribonucleic acid (DNA), cancer cells, imaging scans of cancer lesions and the like. However, not only are these largely inaccurate and inefficient, they are limited to the scope of detecting cancer at the later stages of the disease.

### SUMMARY

The present disclosure provides an improved computerized framework for antigen-independent de novo prediction of cancer-associated TCR repertoire. The disclosed framework is a pan-cancer early detection tool that is able to augment the small signals emitted from early stage cancer by analyzing and understanding the changes in the blood T cell repertoire. The disclosed systems and methods provide for the ability to detect, at the earliest stages, cancers that many current technologies are unable to identify—for example, kidney cancer, ovarian cancer and pancreatic cancer. As discussed herein, in addition to the improved capabilities for early-stage cancer detection, the disclosed framework provides capabilities for improving the accuracy of detecting late-stage cancer in patients, as, for example, it can be used together with radiographic images to increase their diagnostic accuracy (in addition to the existing traditional methods mentioned above).

The disclosed systems and methods embody the first immune-based cancer detection techniques or technology. That is, when an individual has cancer, the immune system will react by proliferation of cancer-specific T cells and circulate them in the blood and lymph system. While this bodily reaction is naturally occurring, its presentation in, and the analysis of blood data is not, and thus an improved automated framework is necessary to perform such analysis. The disclosed framework uses a specific automation technique to detect cancer signals from the signatures of the peripheral immune repertoire, which can be performed with higher accuracy than present automated methodologies even at the early stages of the disease.

According to some embodiments of the instant disclosure, the disclosed framework executes a novel machine learning algorithm that can predict cancer status based on a patient's peripheral blood TCR repertoire. As discussed in more detail below, starting with a normal amount of blood sample (e.g., 3-10 ml), the disclosed framework can perform deep TCR sequencing of the genomic DNA of the white blood cells, which enables the detection (prediction or determination) of cancer-associated TCRs independent of tumor antigens. This is then leveraged in order to identify a patient's “cancer score”, which is reflective of their immune repertoire. The score is an output of an automated process which output represents a robust biomarker for both early and late-stage cancers across diverse diseases, and is predictive of patient response to checkpoint blockade therapies. Thus, the determined score is a strong indicator of whether a patient has cancer, and to what degree.

In accordance with one or more embodiments, the instant disclosure provides computerized methods for a novel framework for diagnosing cancer status with peripheral blood TCR repertoire. In accordance with one or more embodiments, the instant disclosure provides a non-transitory computer-readable storage medium for carrying out the above mentioned technical steps of the framework's functionality. The non-transitory computer-readable storage medium has tangibly stored thereon, or tangibly encoded thereon, computer readable instructions that when executed by a device cause at least one processor to perform a method for a novel and improved framework for diagnosing cancer status with peripheral blood TCR repertoire.

In accordance with one or more embodiments, a system is provided that comprises one or more computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a schematic diagram illustrating an example of a network within which the systems and methods disclosed herein could be implemented according to some embodiments of the present disclosure;

FIG. 2 is a block diagram illustrating components of an exemplary system in accordance with some embodiments of the present disclosure;

FIG. 3A is a schematic diagram illustrating an example data flow of the disclosed systems and methods according to some embodiments of the present disclosure;

FIG. 3B illustrates a non-limiting example embodiment of selected features according to some embodiments of the present disclosure

FIG. 4 depicts is a schematic diagram illustrating a non-limiting data flow of the disclosed systems and methods in accordance with some embodiments of the present disclosure;

FIG. 5A, FIG. 5B and FIG. 5C illustrate non-limiting examples of predicted cancer relevance data in accordance with some embodiments of the present disclosure;

FIG. 6 illustrates a data resource table of training and testing data in accordance with some embodiments of the present disclosure;

FIG. 7 illustrates non-limiting examples of sequence conservation patterns in accordance with some embodiments of the present disclosure;

FIG. 8 illustrates non-limiting examples of biochemical features of TCRs in accordance with some embodiments of the present disclosure;

FIG. 9 illustrates non-limiting examples of ROC curves in accordance with some embodiments of the present disclosure;

FIG. 10 illustrates non-limiting examples of variations of 3-dimensional positions for the −6 residue in accordance with some embodiments of the present disclosure;

FIG. 11A, FIG. 11B and FIG. 11C illustrate non-limiting examples of performance evaluations of cancer scores and Shannon's entropy in accordance with some embodiments of the present disclosure;

FIG. 12 illustrates non-limiting examples of predicting cancer status in accordance with some embodiments of the present disclosure;

FIG. 13A and FIG. 13B illustrate non-limiting examples of random fluctuations of cancer scores in accordance with some embodiments of the present disclosure; and

FIG. 14 illustrates non-limiting examples of distributions of cancer scores for cancer patients in accordance with some embodiments of the present disclosure.

### DESCRIPTION OF EMBODIMENTS

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

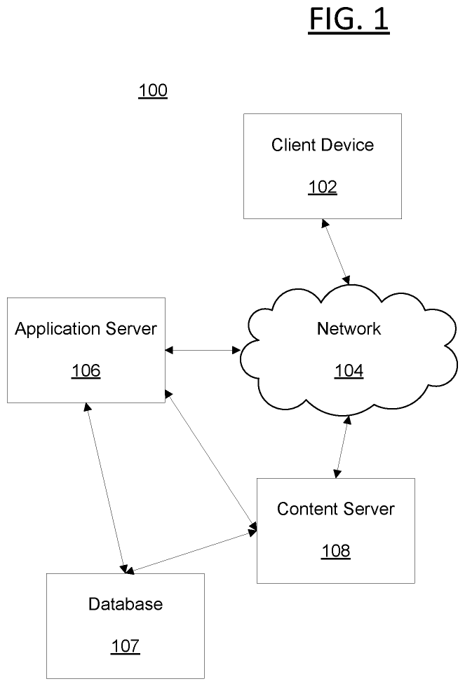

Certain embodiments will now be described in greater detail with reference to the figures. In general, with reference to FIG. 1, a system 100 in accordance with an embodiment of the present disclosure is shown. FIG. 1 shows components of a general environment in which the systems and methods discussed herein may be practiced. Not all the components may be required to practice the disclosure, and variations in the arrangement and type of the components may be made without departing from the spirit or scope of the disclosure.

As shown, system 100 of FIG. 1 includes network 104, which as discussed above can include, but is not limited to, a wireless network, a local area network (LAN), wide area network (WAN), the Internet, or a combination thereof.

Network 104 may be configured to device(s) 102 and its components with another network or device. Network 104 may be configured as a variety of wireless sub-networks that may further overlay stand-alone ad-hoc networks, and the like, to provide an infrastructure-oriented connection for device(s) 102 and servers 106-108. Network 104 is enabled to employ any form of computer readable media or network for communicating information from one electronic device to another.

System 100 also includes device(s) 102, which can be a client device(s). A client device may, for example, include a desktop computer or a portable device, such as a cellular telephone, a smart phone, a display pager, a radio frequency (RF) device, an infrared (IR) device an Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

Device(s) 102 also may include at least one client application that is configured to receive content from another computing device. The device(s) 102 can communicate over the network 104 with other devices or servers, and such communications may include sending and/or receiving messages, generating and providing TCR data, searching for, viewing and/or sharing TCR data, or any of a variety of other forms of communications. Device 102 may be capable of processing or storing signals, such as in memory as physical memory states, and may, therefore, operate as a server

System 100 also includes a variety of servers, such as content server 108, application (or “app”) server 106, and database (for data storage of the processing performed herein) 107.

The app server 106 and content server 108 may include a device that includes a configuration to provide and/or generate any type or form of content via a network to another device. Devices that may operate as app server 106 and/or content server 108 include personal computers desktop computers, multiprocessor systems, microprocessor-based or programmable consumer electronics, network PCs, servers, and the like. It should be understood that servers 106 and 108 can store various types of data related to the content and services provided by servers 106 and 108 in an associated database 107.

In some embodiments, users (e.g., patients, doctors, technicians, and the like) are able to access services provided by servers 106 and 108. This may include in a non-limiting example, application servers, authentication servers, search servers, exchange servers, via the network 104 using their various device(s) 102.

Thus, the app server 106, for example, can store various types of applications and application related information including application data and user profile information (e.g., information determined from or relied upon Process 400, as discussed below, for example).

Moreover, although FIG. 1 illustrates servers 106 and 108 as single computing devices, respectively, the disclosure is not so limited. For example, one or more functions of servers 106 and/or 108 may be distributed across one or more distinct computing devices. Moreover, in one embodiment, servers 106 and/or 108 may be integrated into a single computing device, without departing from the scope of the present disclosure.

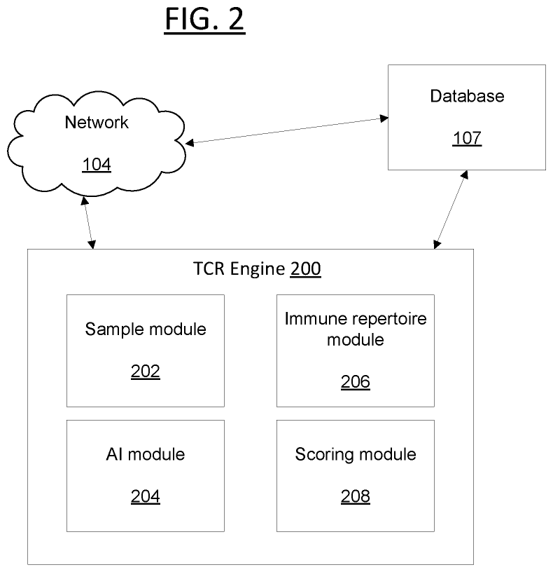

FIG. 2 is a block diagram illustrating the components for performing the systems and methods discussed herein. FIG. 2 includes TCR engine 200, network 104 and database 107. Engine 200 can be a special purpose machine or processor and could be hosted by an application server, content server, web server, third party server, user's computing device, and the like, or any combination thereof.

According to some embodiments, engine 200 can be embodied as a stand-alone application that executes on a device (e.g., a user device or system/web-connected server/device). In some embodiments, the engine 200 can function as an application installed on the device, and in some embodiments, such application can be a web-based application accessed by the device over a network. In some embodiments, the engine 200 can be installed as an augmenting script, program or application (e.g., a plug-in or extension) to another application, such as, for example, a health care application that aggregates and shares patient related data.

The database 107 can be any type of database or memory, and can be associated with a server on a network (e.g., app and content servers 106 and 108) or a user's device (e.g., device(s) 102). Database 107 comprises a dataset of data and metadata associated with local and/or network information related to users, services, applications, content and the like. Such information can be stored and indexed in the database 107 independently and/or as a linked or associated dataset. As discussed herein, it should be understood that the data (and metadata) in the database 107 can be any type of information and type, whether known or to be known, without departing from the scope of the present disclosure.

According to some embodiments, database 107 can store data for users, e.g., user data. According to some embodiments, the stored user data can include, but is not limited to, for example, information associated with a patient's cancer diagnosis, patient's chromosomal information, patient's DNA information, patient's blood information, patient demographic information, patient biographic information, and the like, or some combination thereof.

It should be understood that the data (and metadata) in the database 107 can be any type of information related to a patient, doctor, content, a device, an application, a service provider, a content provider, whether known or to be known, without departing from the scope of the present disclosure.

In some embodiments, the data stored in database 107 can be encrypted, for example using a 256-bit encryption, such that the data is private and controlled according to Health Insurance Portability and Accountability Act of 1996 (HIPPA).

Database 107 can store and index the information in database 107 as linked set of data and metadata, where the data and metadata relationship can be stored as the n-dimensional vector. Such storage can be realized through any known or to be known vector or array storage, including but not limited to, a hash tree, queue, stack, VList, or any other type of known or to be known dynamic memory allocation technique or technology. It should be understood that any known or to be known computational analysis technique or algorithm, such as, but not limited to, cluster analysis, data mining, Bayesian network analysis, Hidden Markov models, artificial neural network analysis, logical model and/or tree analysis, and the like, and be applied to determine, derive or otherwise identify vector information for patients and/or health care providers.

As discussed above, with reference to FIG. 1, the network 104 can be any type of network such as, but not limited to, a wireless network, a local area network (LAN), wide area network (WAN), the Internet, or a combination thereof. The network 315 facilitates connectivity of the engine 200, and the database of stored resources 107. Indeed, as illustrated in FIG. 2, the engine 200 and database 107 can be directly connected by any known or to be known method of connecting and/or enabling communication between such devices and resources.

The principal processor, server, or combination of devices that comprises hardware programmed in accordance with the special purpose functions herein is referred to for convenience as engine 200, and includes sample module 202, AI module 204, immune repertoire module 206 and scoring module 208. It should be understood that the engine(s) and modules discussed herein are non-exhaustive, as additional or fewer engines and/or modules (or sub-modules) may be applicable to the embodiments of the systems and methods discussed. The operations, configurations and functionalities of each module, and their role within embodiments of the present disclosure will be discussed below.

The principles described herein may be embodied in many different forms. T cells reactive to tumor antigens are central mediators of cancer immunity and key targets of immunotherapies, yet as most of the cancer antigens are unknown, experimental detection of cancer-associated T cells remains difficult. The recent development of deep immune repertoire sequencing (TCR-seq) technology has placed an additional emphasis on the identification of such T cells, as it may open new opportunities for non-invasive clinical diagnosis, prognosis and longitudinal immune monitoring of cancer patients.

However, human immune repertoire contains public T cells, naïve T cells, and memory/effector T cells specific to diverse antigens, and this complexity adds to the challenges conventional systems are unable to solve—e.g., to identify cancer-associated T cells in the TCR-seq data.

Previous studies on the TCR repertoires of cancer patients reported that simple statistics, such as diversity and clonality, are associated with clinical outcome under certain conditions, substantiating the utilities of repertoire data as a potential prognostic factor. However, with the fast advancement of immunotherapies and rapid accumulation of TCR-seq data, more computational tools are required to bridge the gap between basic immunogenomics research and clinical applications beneficial to cancer patients.

The disclosed systems and methods provide these needed tools through a novel framework executing ensemble machine learning software (referred to as TCRboost) that provides for de novo prediction of cancer-associated immune repertoires using the β chain TCR-seq data.

According to some embodiments, the disclosed framework utilizes TRUST, an open source algorithm for calling the TCR transcript hypervariable CDR3 regions (complementary determining region 3) using unselected RNA-seq (ribonucleic acid sequence) data profiled from solid tissues. TRUST, as understood by those of skill in the art, has achieved high sensitivity in CDR3 calling even for samples with low sequencing depth and has demonstrated utilities in its application to large tumor cohorts.

While discussion of embodiments discussed herein will focus on utilizing the TRUST algorithm/software, it should not be viewed as limiting, as the disclosed framework can utilize any known or to be known machine learning or artificial intelligence (AI) technique, algorithm or mechanism without departing from the scope of the initial disclosure.

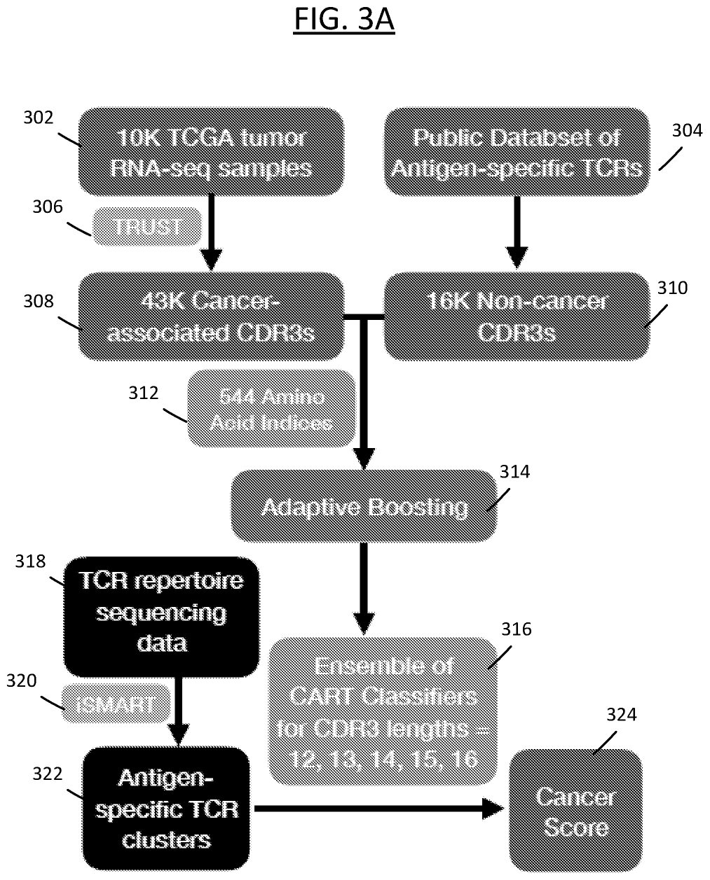

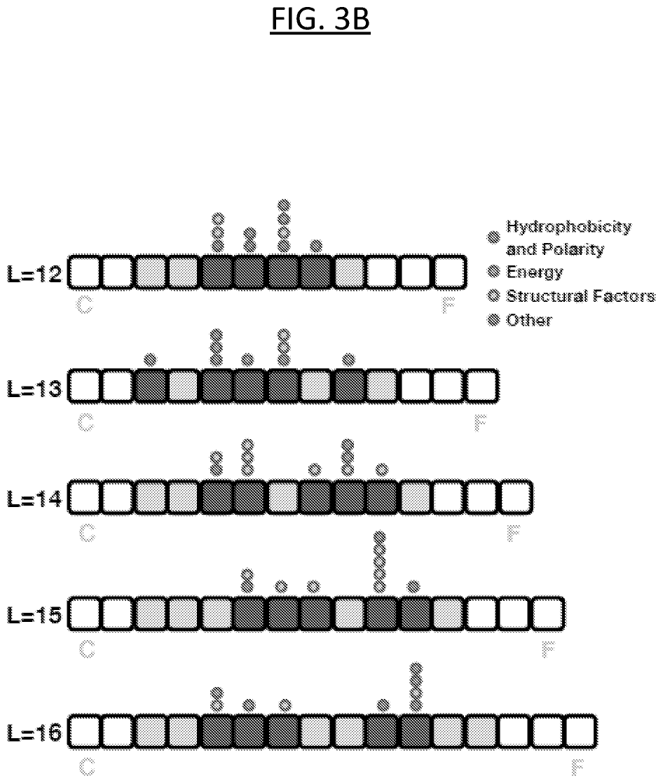

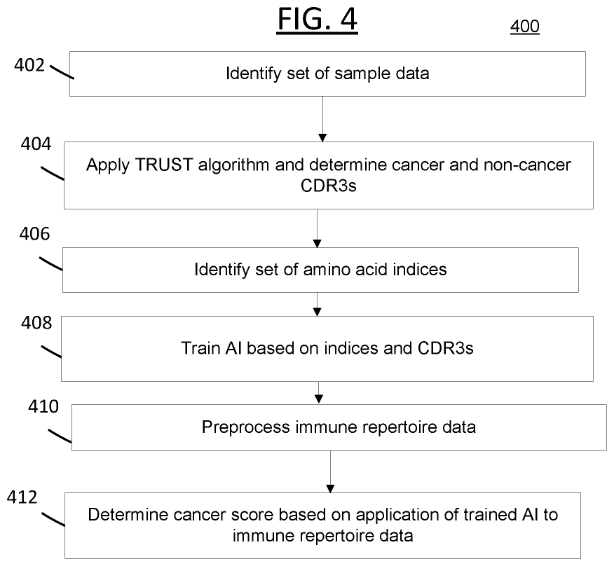

According to some embodiments, the TRUST algorithm is executed in order to analyze a set of (e.g., 10,000) TCGA (The Cancer Genome Atlas) tumor samples covering a predetermined number (e.g., 32) cancer types; and as a result, a number of non-public complete productive βCDR3 sequences are collected/determined (e.g., 43,000 non-public complete productive βCDR3 sequences). This is discussed in more detail below, in reference to FIG. 3A and FIG. 4.

According to some embodiments, TRUST-called CDR3s are enriched for expanded clonotypes, and thus likely to be tumor-associated. In addition, as the βCDR3s come from diverse cancer types, they are unlikely to be biased towards a few cancer antigens.

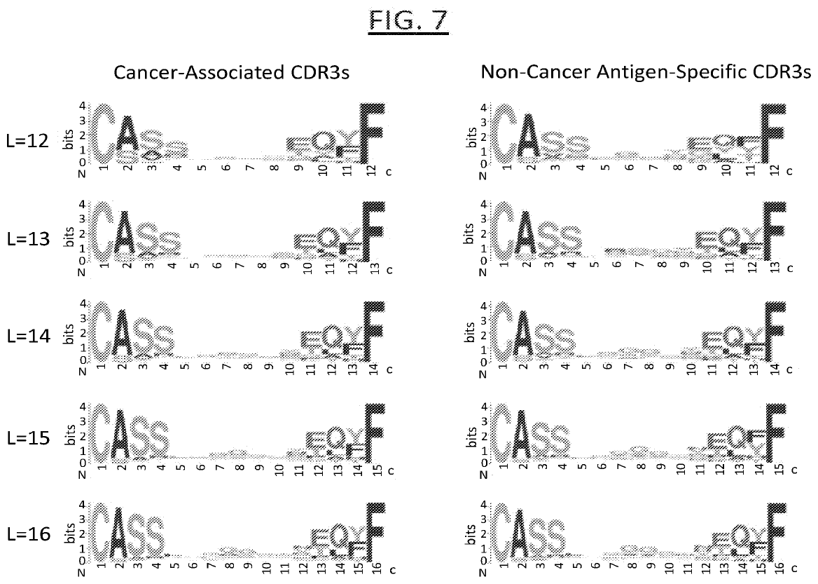

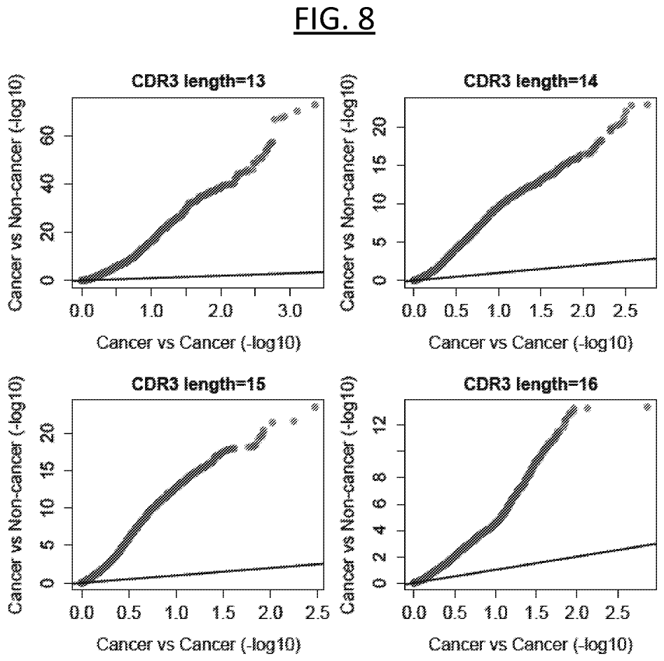

Turning to FIG. 7 and FIG. 8, FIG. 7 illustrates sequence conservation patterns between cancer or non-cancer associated CDR3s with lengths ranging from 12-16, where CDR3 amino acid sequences for each category were analyzed for conservation patterns.

FIG. 8 depicts biochemical features of cancer-associated TCRs showing significant differences from non-cancer TCRs. For CDR3s with length L, the 544×(L−5) features were compared between cancer and non-cancer associated TCRs, with statistical significance evaluated using two-sided Wilcoxon rank sum test. As control, cancer-associated TCRs were randomly split into two groups, between which p values for each feature were estimated. The cancer vs non-cancer p values were compared with the cancer vs cancer ones on quantile-quantile (Q-Q) plots (−log values), where the formers are significantly higher than the latter consistently for all CDR3 lengths.

Thus, although there are no apparent differences in sequence conservation patterns between cancer or non-cancer CDR3s (FIG. 7), significant differences in the amino acid indices were observed (FIG. 8), which evidences distinctive biochemical signatures for cancer-associated TCRs.

Therefore, the βCDR3 sequences derived from the TCGA data can serve as a valid training dataset for cancer-associated TCRs.

According to some embodiments, the framework applies a machine learning meta-algorithm, such as, for example, Adaptive Boosting (AdaBoost). As understood by those of skill in the art, AdaBoost reduces the speed in training and executing a classifier of an AI system by selecting and training only those features that are known to improve the predictive power of the model, thereby reducing the dimensionality while improving the execution time.

While discussion of some embodiments discussed herein will focus on utilizing AdaBoost, it should not be viewed as limiting, as the disclosed framework can utilize any known or to be known machine learning or artificial intelligence (AI) technique, algorithm or mechanism without departing from the scope of the initial disclosure. That is, as discussed in more detail below (e.g., in reference to FIG. 3A and FIG. 4), in addition to or in the alternative to AdaBoost, any known or to be known type or form of machine learning/AI can be utilized to analyze T cell, blood or tumor samples/types in a similar manner—such as, but not limited to, Artificial Neural Networks (ANN), Deep Neural Networks (DNN), Convolutional Neural Networks (CNN), and the like.

According to some embodiments, AdaBoost is applied to train an ensemble tree classifier to distinguish cancer-associated TCRs from non-cancer ones. In some embodiments, the application occurs separately for CDR3s with length=12, 13, 14, 15 and 16. The performance of the classifier in predicting tumor-reactive CDR3s was evaluated using cross-validation.

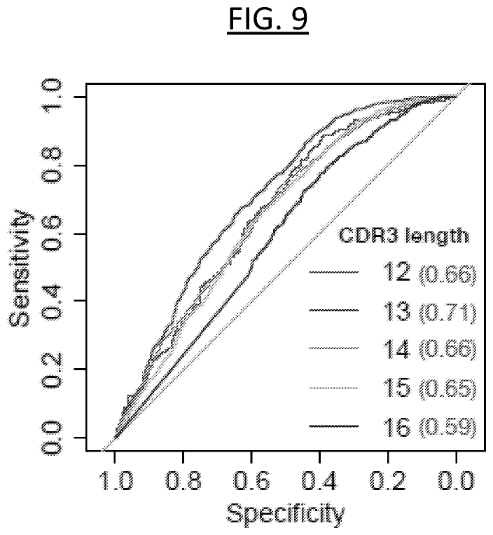

As measured by area under ROC (receiver operator curve) (AUROC), the prediction power is highest for CDR3 length=13 (AUROC=0.71). This is illustrated in FIG. 9, where ROC curves measuring the prediction power for individual cancer-associated CDR3s with different lengths is depicted. Ensemble tree classifiers for each CDR3 length were applied to the testing data in four-fold cross validation analysis. For each CDR3, the classifier predicted a probability of its being cancer-associated. Using the probability as a continuous parameter, ROC curves were generated, with AUROC labeled in the figure. Features with top classification power are displayed at each amino acid location in the CDR3 loop, with the −6 position having the highest number of hits (as illustrated in FIG. 3B and discussed below).

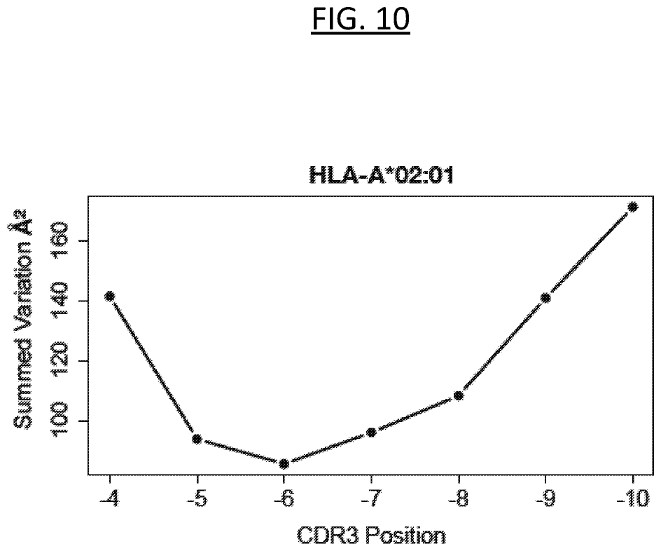

Analysis of selected TCR/pMHC structures provide that this position is at the intersection of antigen, MHC-I α1 helix, and TCR a chain. The coordinates of the −6 position Cα have the lowest variation in the 3D space (as illustrated in FIG. 10, where analysis was performed using HLA-A*02:01 binding antigens and T cell receptors), indicating its structural conservation. These results provide that the trained AdaBoost classifier (and/or deep neural classifier) captures biochemical signatures that are potentially important in TCR/pMHC interaction.

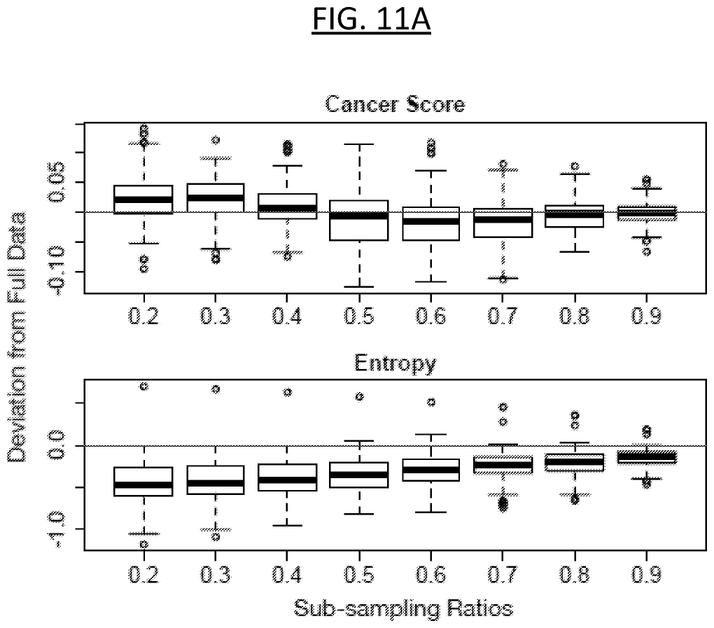

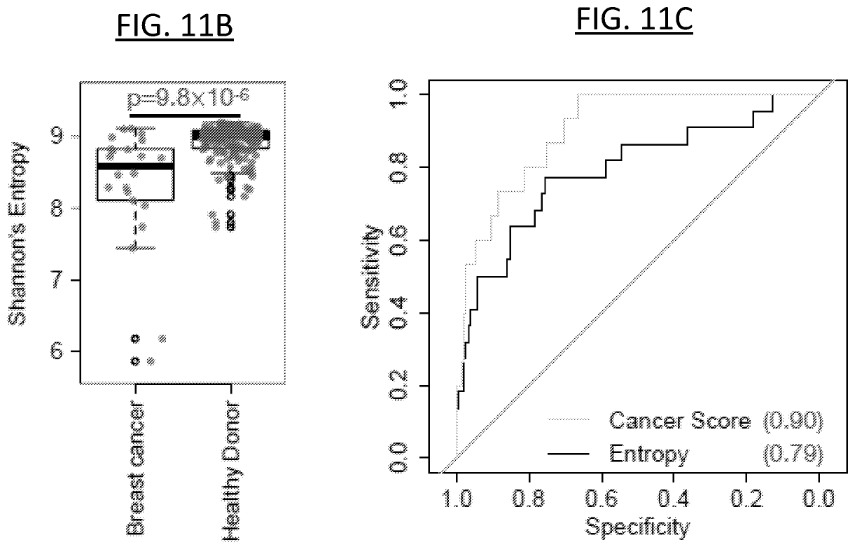

For a given TCR repertoire data, the most abundant clonotypes are grouped into highly specific clusters. The tree classifier is then applied to each of the clustered CDR3s to predict the probability of being cancer-associated. The outcomes are aggregated into a cancer score ranging from 0 to 1. Unlike Shannon's entropy, the disclosed approach is almost invariant to sequencing depth, making the cancer score estimations directly comparable between different studies. This is illustrated in FIG. 11A, which depicts the results of subsampling analysis showing that a cancer score is robust to variable sequencing depths, where entropy monotonously decreases with lower depths.

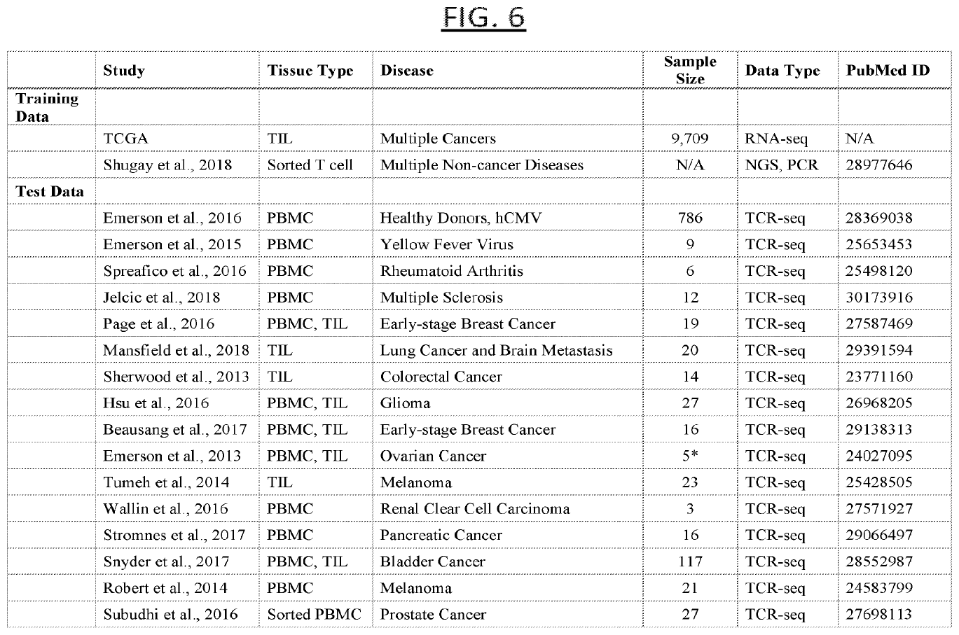

By way of a non-limiting example, illustrating the accuracy and efficiency the disclosed framework, 16 independent public TCR-seq sample cohorts were analyzed to systematically evaluate the performance of TCRboost, as illustrated in the Table of FIG. 6.

FIG. 6 provides a summary of datasets used for training and testing purposes. Training data was derived from TCRs extracted from tumor RNA-seq data of TCGA samples, and T cells specific to non-cancer antigens from literature. Testing data came from 16 sample cohorts in the public domain, with sample size and pubmed IDs labeled. *: for the ovarian cancer cohort, multi-section sampling was performed on tumors from 5 patients, and each TIL sample was used as an independent observation.

To explore the behavior of cancer scores in non-cancer patients, TCRboost was applied to a cohort of healthy donors with no major diagnosed diseases, and the cancer scores of this cohort is used as a baseline. Peripheral Blood Mononuclear Cell (PBMC) samples from 4 cohorts of non-cancer conditions were utilized, which included chronic HCMV (human cytomegalovirus) infection, yellow fever virus vaccination, rheumatoid arthritis and multiple sclerosis.

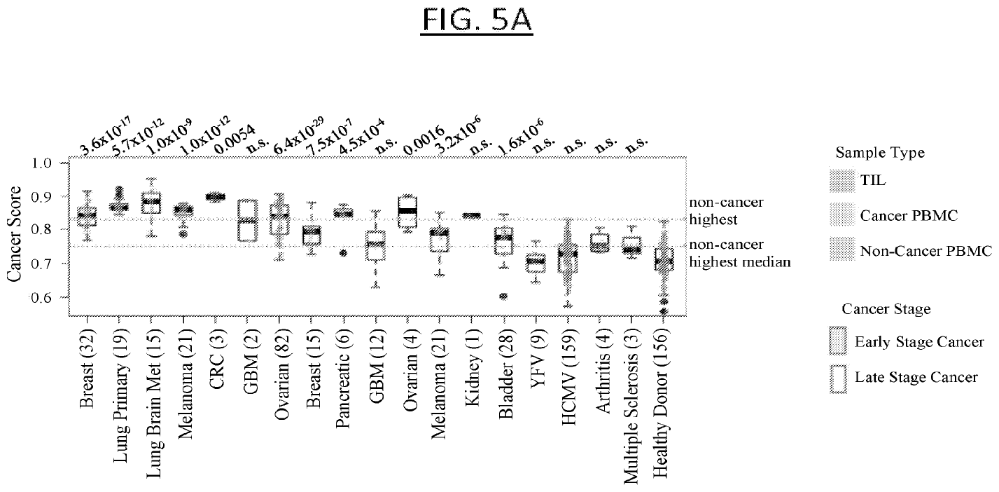

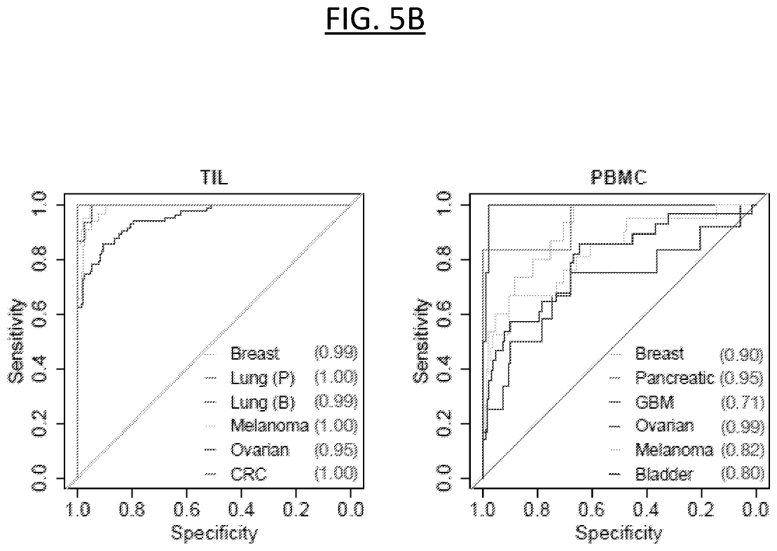

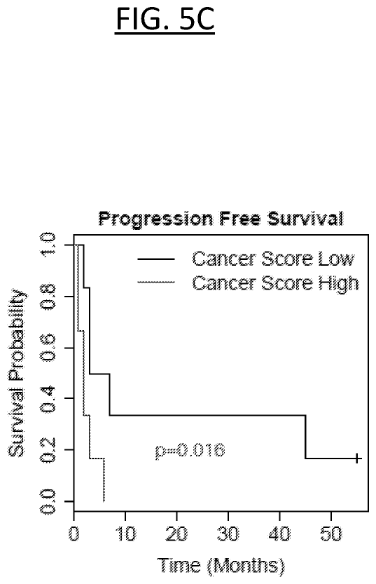

As illustrated in FIG. 5A, cancer scores of none of the above cohort showed significant deviation from baseline at FDR=0.01. FIG. 5A illustrates cancer score distributions across diverse disease and tissue types displayed by boxplots, with original data overlaid as transparent red points. Numbers in the parenthesis in the x-axis label are sample size for each cohort. Two-sided Wilcoxon rank sum test was performed between each cohort and the scores of healthy donors, with Benjamini-Hochberg corrected FDR levels were displayed on top of each box.

TCRboost was then applied to PBMC or tumor-infiltrating T lymphocyte (TIL) repertoires of patients with diverse cancer types, including breast, brain, ovarian, pancreatic, bladder, kidney, colorectal, non-small cell lung cancers and melanoma. The cancer scores of most cohorts are significantly higher than healthy donors (as illustrated in FIG. 5A), except for kidney cancer due to small sample size, and for glioblastoma (GBM), which is likely due to limited T cell infiltration and reduced antigen presentation in the brain tissue. Generally higher cancer scores for TIL repertoires than those for PBMCs are evident, potentially because cancer-associated T cells are enriched in TILs. These results indicated that TCRboost predicted scores are specifically higher in cancer samples, and can distinguish patients of multiple cancer types from healthy individuals.

Thus, the determined cancer score can be a single predictor for cancer status.

By way of a non-limiting example, for each cancer cohort, the scores were mixed with those from healthy donors, and generated the ROC curves to measure sensitivities and specificities, as illustrated in FIG. 5B. FIG. 5B illustrates ROC curves measuring the prediction powers of cancer scores as a single variable for cancer status, respectively for TIL samples (left) and PBMC samples (right). For both tissue types, PBMC repertoires of healthy donors were used as control. Area under ROC curves (AUROC) for each cohort were labeled in the parenthesis in the figure legends. Lung (P) is for primary lung tumor, and Lung (B) is for lung tumor brain metastasis.

For TIL samples, cancer scores reached nearly prefect prediction power (AUROC≥0.95) for all cohorts with sufficient sample size (n≥3). For PBMC samples, prediction powers are high for breast, pancreatic and ovarian cancers, medium for melanoma and bladder cancer, and low for GBM. Importantly, the breast cancer samples in the above analysis came from two early-stage breast cancer cohorts, and an AUROC of 0.99 (99%) can be observed. After subsampling, entropy can also distinguish early breast cancer from healthy donors, but the prediction power is substantially worse (AUROC=0.79), as illustrated in FIG. 11B and FIG. 11C.

FIG. 11B illustrates entropy calculated from PBMC repertoire samples of early breast cancer patients that is significantly lower than from healthy donors. Both cohorts were down-sampled to 10,000 reads before comparison. FIG. 11C illustrates the performance of entropy as a predictor for early-stage cancer is substantially worse than the disclosed cancer score.

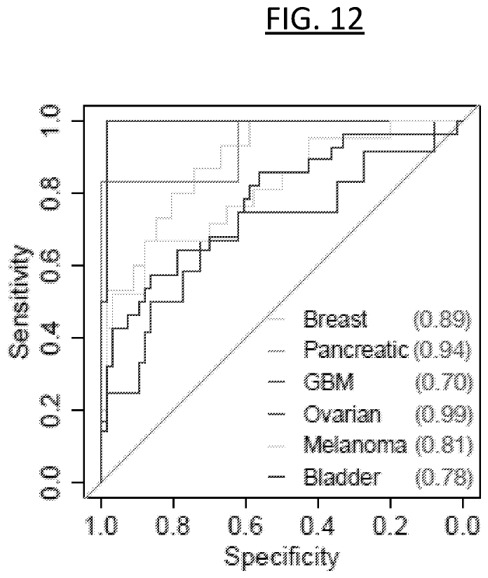

At cut-off of 0.75, cancer score reaches 80.0% sensitivity, and 81.4% specificity. This performance is better than many existing cancer screening approaches. This analysis can be repeated using another control cohort of PBMC samples from healthy donors, and as illustrated in FIG. 12, very similar ROCs can be observed.

Therefore, based on the high prediction powers, the cancer scores can be used to detect cancer-associated blood TCR repertoires.

The disclosed adaptive immune repertoire is a dynamic system that provides accurate cancer scores. Despite the random fluctuations of the immune repertoire, a healthy donor would not have a cancer score as high as cancer patient (e.g., the disclosed system avoids the changes of false-positives for a cancer diagnosis).

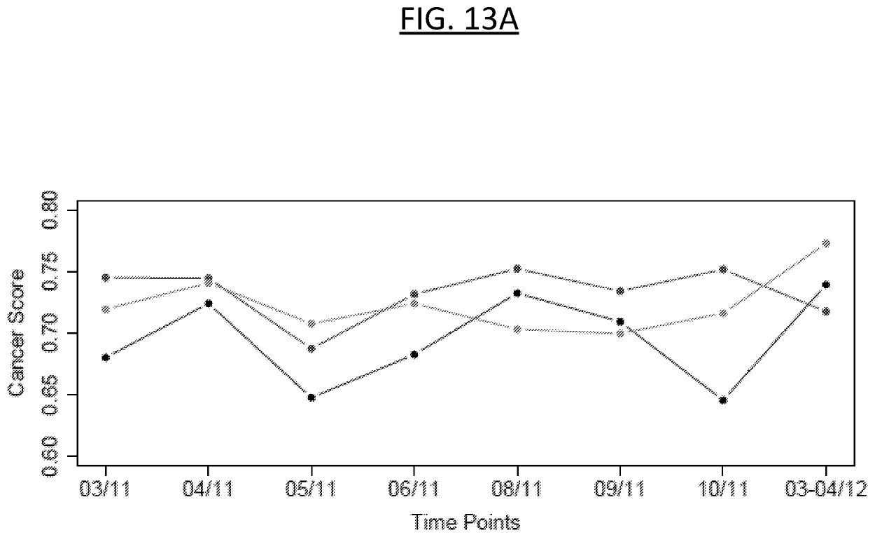

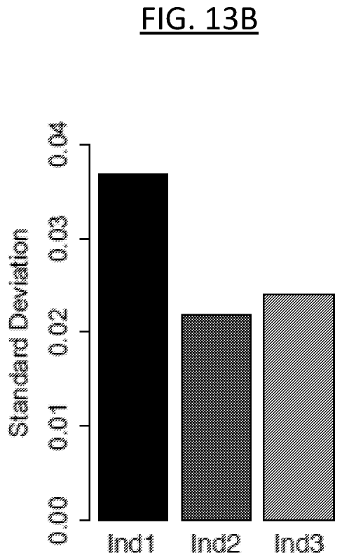

For example, the random fluctuations of cancer scores of PBMC samples from healthy donors were evaluated over one year of time. Of the three individuals examined, it was observed that relatively small longitudinal changes of scores (as illustrated in FIG. 13A), with standard deviations <0.04 for all individuals (as illustrated in FIG. 13B, which is a bar-plot showing the standard deviations calculated from the time points of each individual). The mean score for healthy donors is 0.71, and for early stage breast cancer patients is 0.79, which is more than 2 standard deviations higher than healthy donors. Therefore, it is unlikely for a healthy donor to have cancer score as high as cancer patients due to random fluctuations in the immune repertoire, and vice versa.

Prediction of cancer immunotherapy response is currently of great clinical interest. FIG. 5C depicts Kaplan-Meier curves showing significant survival differences between two groups of melanoma patients with BRAF mutations treated with Ipilimumab. The group with better outcome has lower cancer scores in their pre-treatment PBMC samples. P value was evaluated using Cox proportional hazard model controlled for patient age and Shannon's entropy. P value for age or entropy was insignificant.

FIG. 5C depicts the determined cancer scores of TCR-seq samples from two patient cohorts treated with immune checkpoint blockade (ICB). Interestingly, for melanoma patients with BRAF mutations treated with Ipilimumab, an anti-CTLA4 mAb (monoclonal antibody), higher cancer score derived from pre-treatment PBMC samples significantly predicts worse outcome. The second cohort was analyzed investigated were metastatic prostate cancer patients treated with Ipilimumab.

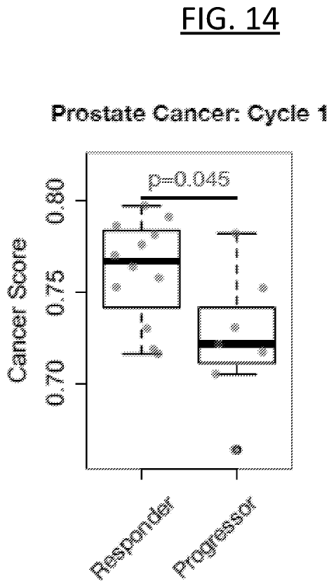

Cancer scores for CD8+ T cells in the PBMC samples after the first cycle of treatment are significantly higher in the responders than progressors (as illustrated in FIG. 14). These results suggest that PBMC cancer scores may help to monitor patient outcome in anti-CTLA4 immunotherapy.

Thus, in summary, the instant disclosure provides for the detection of a novel biochemical signature of cancer-associated TCRs from tumor genomics sequencing data, which is independent of tumor antigens as well as patient HLA allelotypes. It is reproducibly observed in the TCR-seq sample cohorts of diverse cancer types. TCRboost aggregates many TCRs in a repertoire to estimate the cancer scores, which are significantly higher for cancer patients and robust to random fluctuations, making it a legitimate candidate for non-invasive diagnostic biomarker.

In addition, as cancer scores are predicted from the immune system, it is orthogonal to most contemporary detection methods based on cancer biomarkers, imaging scan or circulating tumor cell (CTC)/circulating tumor DNA (ctDNA). The cancer scores, therefore, provide predictions that are robust—e.g., they are valid and can withstand and account for −random fluctuations of TCR repertoire over time, thereby providing an accurate indication of whether a patient has cancer and his/her cancer status (e.g., what degree of cancer).

Therefore, contingent use of cancer scores on existing methods is expected to increase cancer detection accuracy and improve clinical decision-making. As cancer scores derived from certain late-stage cancers are associated with patient response to ICB, it may also be used to improve the prediction of clinical outcome of these cancer types. One of skill in the art would understand and anticipate broad utilities of TCRboost in cancer diagnosis and immunotherapy prognosis with the rapidly accumulating TCR repertoire sequencing data in the clinical studies.

Turning to FIG. 3A, a schematic illustration of an embodiment of the TCRboost methodology is provided. Specifically, FIG. 3A depicts a general workflow of the TCRboost processing discussed herein, and FIG. 4 provides the details of each step (which is discussed in more detail below).

In some embodiments, as discussed above, CDR3s are trained either from unselected tumor RNA-seq data (Step 302), or from experimentally determined TCRs specific to various non-cancer antigens (Step 304). Such training is performed, according to some embodiments, via the TRUST algorithm—Step 306. Thus, Step 302 results in the determination of cancer-associated CDR3s (Step 308), and Step 304 results in the determination of non-cancer CDR3s (Step 310). Features for CDR3 regions are defined as the amino acid indices for each position of interest (Step 312), and ensemble tree classifiers are then trained for CDR3s with different lengths using the AdaBoost algorithm (or other supervised machine learning methods, including the deep neural network models), as discussed above and in more detail below. Steps 314-316. Each TCR-seq sample was pre-processed (Step 318), and clustered by immuno-similarly measurement (iSMART) (Step 320) to identify antigen-specific groups (Step 322). Then trained tree classifiers (e.g., trained from Step 314) are applied to the grouped CDR3s to evaluate a cancer score, related to the probability of an immune repertoire being cancer-associated (Step 324).

iSmart involves performing pairwise alignment of CDR3 sequences, then determining scores based on the alignments. Then, building a connectivity matrix of CDR3 sequences based on “high” alignment scores (e.g., scores above a predetermined threshold), where CDR3 clusters are then determined and formed based therefrom. Thus, iSmart (and similar algorithms, as discussed below) can group TCRs into antigen-specific clusters.

One of skill in the art would understand that while the disclosure herein, in FIG. 3A, references the usage of iSMART, it should be viewed as limiting, as any known or to be known form of Markov, semi-Markov decision or reinforcement learning (RL) processes, algorithms, techniques can be employed by the disclosed framework without departing from the scope of the disclosed systems and methods.

FIG. 3B illustrates the locations of CDR3 sequences with lengths ranging from 12 to 16 amino acids. For each length, the most important features for classification were selected and displayed on the corresponding locations (as discussed below in relation to FIG. 2). Each location is represented by a shaded square, with non-shading (e.g., no-shading) indicating positions not covered in the analysis, light-grey for analyzed yet no feature was found important, and dark-gray for locations with important features in classification.

Turning to FIG. 4, Process 400 provides a detailed view of the TCRboost methodology discussed herein. According to some embodiments, Process 400 provides an immune-based cancer detection methodology that can detect cancer signals from the signatures of the peripheral immune repertoire, which can be performed with high accuracy even at the early stages of the disease. An improved framework is employed that is embodied through a novel machine learning algorithm that can predict cancer status based on a patient's peripheral blood TCR repertoire, such that a deep TCR sequencing of the genomic DNA of the white blood cells is performed, which enables the detection (prediction or determination) of cancer-associated TCRs independent of tumor antigens. This provides a robust biomarker for both early and late-stage cancers across diverse diseases.

According to some embodiments of Process 400 of FIG. 4, Step 402 of Process 400 is performed by the sample module 202 of engine 200; Steps 404-408 are performed by AI module 204; Step 410 is performed by immune repertoire module 206; and Step 412 is performed by scoring module 208.

Process 400 begins with Step 402 where a set of sample data is identified, as discussed above in relation to Steps 302-304 of FIG. 3A. In some embodiments, TCGA level 2 BAM files aligned to hg19 human reference genome by MapSplice for tumor gene expression can be downloaded from GDC legacy archive, and processed by TRUST to extract the TCR CDR3 sequences. Other validated approaches can also be used to generate the true positive cancer associated TCRs. In some embodiments, TCR repertoires specific to non-cancer antigens can also be downloaded from VDJdb, for example, or from the blood TCR-seq data of healthy donors in the public domain. In some embodiments, TCR repertoire sequencing data from 14 study cohorts (see FIG. 4) can be downloaded from AdaptiveBiotechnology ImmuneAccess online database.

In Step 404, the TRUST algorithm is applied to these identified samples to determine cancer and non-cancer CDR3s, as discussed above in relation to Steps 306-310 of FIG. 3A. According to some embodiments, the TCGA-derived CDR3s can be filtered in for complete sequence starting with the last cysteine (C) from the variable gene, and the phenylalanine (F) in the FGXG motif in the joining gene. The non-productive sequences containing stop codon between C and F can be excluded. To remove public TCRs that are also found in non-cancer individuals, the top most abundant CDR3s from a cohort of PBMC repertoire samples (e.g., the CDR3s satisfying a threshold—for example, the top 5,000 from 666 healthy or HCMV infected patients) can be collected and filtered out from the set. The resulting CDR3 sequences (e.g., 43,000 CDR3) are expected to be non-public and cancer associated.

In Step 406, a set of amino acid indices are identified, as discussed above in relation to Step 312 of FIG. 3A. The current amino acid index database documented 544 biochemical indices, which can be used as surrogates of the functional and structural impact for amino acids. From the above non-public cancer associated data, CDR3 sequences with length L between 12 and 16 amino acids (AA) are selected, and the first 2 and the last 3 AAs are removed without structural contact to the pMHC complex. The total feature set is union for each informative AA, e.g. the number of features is (L−5)×544. n_{L} is used to denote the number of CDR3s with length L for cancer CDR3s (derived from TCGA data), and k_{L} the number for non-cancer CDR3s (from VDJdb).

In Step 408, the AI algorithm (AdaBoost or deep learning) is trained, as discussed above in relation to Step 314 of FIG. 3A. According to some embodiments, the first 50% of all the sequences from both populations (from Step 202) are sub-sampled, and the remaining half of data is used for cross validation. For each feature, the 0.5 n_{L} cancer observations are compared with the 0.5k_{L} non-cancer ones. If the fold change (cancer over non-cancer) was smaller than 1.1, this feature was removed. Let S denote the number of features left.

In the above setting, there is a total of 0.5×(n_{L}+k_{L}) CDR3 sequences (samples), and S features, with known sample labels (0.5 n_{L} with label 1, and 0.5k_{L} with label −1). Let Y denote the sample label vector with length 0.5×(n_{L}+k_{L}), and X denote the feature matrix with dimension 0.5×(n_{L}+k_{L})-by-S. Based on this analysis, it is determined that the prediction power for individual features is weak.

Therefore, according to some embodiments, AdaBoost can be applied, which, as discussed above, is an ensemble learning approach that is able to aggregate weak classifiers into a stronger one.

Under the AdaBoost embodiments, AI model 204 training is completed using adaboost( ) function in R package JOUSBoost, with 50 rounds of boosting and tree depth of 10. Selected parameters are based on the criteria of minimizing the number of training cycles (rounds) and the complexity of classification tree (depth) while minimizing cross-validation (CV) errors. CV errors are calculated by applying the trained classifier for CDR3 length L (denoted as T_{L}) to the independent validation data with known class labels.

For example, 10 subsampling rounds can be performed, where the best cross validation value is then selected. The above procedure was repeated for L=12, 13, 15 and 16, except for L=14, where four-fold cross validation was applied, as this setting achieved smaller CV error. Therefore, in some embodiments, Step 408 can involve a training of a total of 5 classifiers, according to this example, which are denoted as T₁₂₋₁₆.

According to some embodiments, rather than utilizing AdaBoost, the disclosed framework can train the AI module 204 as a deep neural network. According to some embodiments, for example, the disclosed deep learning methodology employs CNNs (however, it should not be construed to limit the present disclosure to only the usage of CNNs, as any known or to be known deep learning architecture or algorithm is applicable to the disclosed systems and methods discussed herein). CNNs consist of multiple layers which can include: the convolutional layer, rectified linear unit (ReLU) layer, pooling layer, dropout layer and loss layer, as understood by those of skill in the art. When used for CDR3 discovery, recognition and similarity, CNNs produce multiple tiers of deep feature collections by analyzing small portions sample/training data that can be utilized to train a classifier(s).

Thus, according to these embodiments, neural network implementation via Step 408 (and Step 314 of FIG. 3A) can provide a more efficient, accurate system that leverages the processing power and resource expenditure of deep belief networks, in a similar manner as meta-algorithms, as discussed above. Thus, for example, one of skill in the art would understand that neural networks can be utilized to train tree classifiers T₁₂₋₁₆.

In Step 410, immune repertoire data is preprocessed, as discussed above in relation to Steps 318-322 of FIG. 3A. Immune repertoire sequencing data usually contains the DNA and amino acid sequences of the CDR3 region, TCR variable gene, joining gene, and sometimes diversity gene solved by certain callers, and the frequencies of T cell clonotypes (as of CDR3s) in the data. In some embodiments, all the TCR-seq data are generated by AdaptiveBiotechnology immuneAnalyzer, and was focused on the preprocessing steps of the format generated by such processing, though it would be understood by those of skill in the art that the rationale is the same for other file formats as well.

In some embodiments, the following types of low quality calls for CDR3 AA sequences can be removed: 1) sequence length is <10 or >24; 2) sequence contains non-standard characters (*, +, X); 3) sequence is not starting from C or not ending with F; 4) variable gene is not solved. After removal of low quality calls, the remaining CDR3s are decreasingly ordered by clonotype frequencies, and the following columns are selected for clustering analysis: CDR3 amino acid, variable gene and clonotype frequency. For each repertoire data, a predetermined number of sequences satisfying a threshold are selected (e.g., the top 10,000 sequences are selected). If the data contains fewer than 10,000 CDR3s, all will be selected. The cut-off is set to include most of the high abundant clonotypes that are likely to be effector/memory cells, while excluding low frequency naïve cells. Inclusion of excessive number of naïve cells will result in increased noise level, as naïve T cells might be tumor-specific (inactivated) in healthy individuals.

iSMART, a previously developed software solution, is configured to detect antigen-specific T cell groups by clustering CDR3s based on their sequence similarity. Antigen-specificity is based on the recent research on T cells with similar CDR3 motifs are likely to recognize the same antigen. iSMART is shown to have achieved higher specificity than previous methods, benchmarked using TCR sequences specific to different antigens. Thus, iSMART is applied to the pre-processed TCR repertoire sequencing data. The clustering uses both CDR3 sequence and variable gene information to ensure high specificity. Therefore, each of the resulting CDR3 cluster is expected to be responsible for a unique antigen.

In Step 412, a determination (or calculation) of the cancer score is performed, as discussed above in Step 324 of FIG. 3A. According to some embodiments, tree classifiers T₁₂₋₁₆ are applied to the clustered CDR3s. For each TCR with length 12≤L≤16, a score ranging from 0 to 1 is returned, using length-specific tree classifier derived from the step above. The score is the probability of the TCR being cancer-specific. For each length, the scores are aggregated by taking the mean of all the CDR3s with the same length. As a result, five scores are obtained, and the final cancer score is the mean of the five values.

Further Implementation of Disclosed Framework According to Some Non-Limiting Embodiments

According to some embodiments, it is possible that a TCR cluster contains several CDR3s with identical sequences. This is due to the degeneracy of DNA to protein where different TCRs are selected to antagonize the same antigen. They are still counted as different TCR samples.

Additionally, different clusters may have variable sizes, e.g., number of TCRs. Therefore, the score for each TCR can be calculated, disregarding which cluster it belonged to.

In some embodiments, if a repertoire does not contain enough data, for example, clustered CDR3s with certain length was missing, it is reported NA in the final score. This situation usually occurs for TIL samples where few T cells are collected for sequencing. For PBMC repertoires with deep coverage, there are usually enough data to make estimations.

Selection of Representative Features from Classification Trees

According to some embodiments, each classifier contains a predetermined number (e.g., 50) classification and regression trees (CART). Each CART is a binary decision tree with trained thresholds of certain feature at each node. In order to evaluate which feature(s) are important in the classifications, a decrease in deviance is utilized, which is a measure of classification errors. For example, for each tree, features with deviance decrease ≥0.002 are selected. Pooling all the selected features from 50 trees, the frequencies for each recurrent feature can be counted. For example, features with top 10 frequency counts are selected for display in FIG. 1B.

Analysis of TCR/pMHC Protein Complex Structural Data

128 pdb files were downloaded for structures with HLA-A2 allele from rcsb.org on Sep. 12, 2018. HLA-A2 allele was analyzed because it has the largest sample deposit on PDB. Structures that do not contain both TCR and antigen peptide were removed. For each of the 30 remaining structures, the coordinates of the Cα of histidine at the 151^{st} position of the HLA heavy chain as origin was used. This analysis is based on the experimental observation that the structure of HLA heavy chain stabilizes when binding to different TCRs and antigen peptides. The Ca coordinates for β chain CDR3 amino acid located at −4, −5, −6, −7, −8, −9 and −10 positions relative to the phenylalanine located at the end of CDR3 sequence were identified. The Euclidean distances between origin and each of the CDR3 Cα positions were calculated across all the structures. Standard deviation of the distance for each of the positions was then calculated and displayed. Visualization of selected PDB structures for the −6 position of the β chain CDR3 region was performed using Chimera and PyMol.

Post-Processing of Cancer Scores from TCR Repertoire Data and ROC Analysis

As each cohort of TCR-seq samples are designed differently, a consensus approach to select the PBMC and TIL samples to maximize comparability was applied. As in FIG. 4, the Emerson et al., 2015 cohort for yellow fever virus has day 1 and day 14 samples post vaccination on healthy volunteers, and a day 14 sample was used because they are expected to further differ from healthy donors. PBMC samples of whole blood are used for rheumatoid arthritis and multiple sclerosis patients.

For cancer cohorts with longitudinal samplings, including Page et al., 2016, Tumeh et al., 2014, Robert et al., 2014 and Snyder et al., 2017 (from FIG. 4), TIL or PBMC samples were used that were either subject to pre-treatment, or the first cycle after treatment if pre-treatment samples are not available. The samples from the two early breast cancer cohorts were merged (Page et al., 2016 and Beausang et al., 2017) in the analysis.

A calculation of the median differences of cancer score values between each diseased cohort and healthy donors was performed, and an evaluated statistical significance determination was performed using Wilcoxon rank sum test; and, corrected p values were used via Benjamini-Hochberg (BH) procedure, with cut-off false disclover rate (FDR)=0.01 for significance. To evaluate the prediction power of cancer scores, the scores for each cohort with sample size greater than or equal to a predetermined number (e.g., n ≥5) were pooled, with healthy donors, and used function roc( ) in R package pROC to calculate area under curve and make the ROC plots.

Subsampling and Prediction of Cancer Status with Shannon's Entropy

In order to explore the impact of read depths on the estimation of cancer scores and Shannon's entropy, an in silico subsampling analysis was conducted. In some embodiments, a random sampling of 100 individuals from the 666 healthy or HCMV infected individuals was performed. For each TCR-seq data, the same pre-processing procedures described above to remove non-productive, low quality CDR3 calls was performed. The filtered data contains read count (nᵢ) for each CDR3 i, and a new dataset G can be construed by repeating CDR3 i for nᵢ times.

The number of rows of G is the summation of all the read counts in the filtered data. A sampling of 20%, 30%, 40%, 50%, 60%, 70%, 80% and 90% of the rows of G can be performed, with each row representing a sequencing read. That is, in the TCR repertoire sequencing, one read is sufficient to cover one CDR3 region. Therefore, sequencing read counts as CDR3 counts for each clonotype can be used. For each of the subsampled data, re-calculations of the frequencies of each CDR3 can be performed, which result in the generation of a smaller TCR-seq dataset with reduced sequencing depth. Shannon's entropy was estimated using this dataset, while a top threshold satisfying number (e.g., a top 10,000) of most frequency clonotypes for estimations of cancer scores can be selected. The differences of scores between each sequencing depths (represented by sampling ratios) and those of the full datasets are then displayed as boxplots in FIG. 9A.

Shannon's entropy also has some statistical power to distinguish immune repertoires associated with cancer patients and those from healthy individuals. Therefore, it was examined as to whether entropy can also be used as a predictor for early-stage cancer onset. Since entropy is systematically biased by sequencing depths, all PBMC TCR repertoire data was down-sampled for early stage breast cancer and healthy donors to 10,000 reads using the above method. Entropy for each of the down-sampled file was calculated and compared between breast cancer and healthy individuals. Two-sample test and ROC analysis are performed in the same way as for cancer scores. Shannon's entropy was calculated using R package entropy.

Statistical Analysis

All statistical analyses were performed using R the statistical programming language. Two sample tests were performed using two-sided Wilcoxon rank sum test. If multiple tests were performed for a single analysis, BH procedure can be used to correct for FDR, except for FIG. 5, as the purpose was to compare distributions of p values, instead of reporting significance. For all the boxplots displayed in the figures, the middle line defines the median value, with borders of the boxes indicating the 25% (Q1) and 75% (Q3) quartiles of the data. Lower and upper whiskers corresponded to Q1-1.5IQR and Q3+1.5IQR, where IQR is short for inter-quartile range. Survival analysis in FIG. 3C was carried out using R package survival, with p value evaluated using Cox proportional hazard model corrected for patient age.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising the steps of:
identifying, via a computing device, a set of ribonucleic acid sequence (RNA-seq) data;
identifying, via the computing device, data associated with a set of antigen-specific T cell receptors (TCRs);
analyzing, via the computing device executing an algorithm for calling a TCR transcript hypervariable complementary determining region 3 (CDR3 regions), said RNA-seq data and said TCR data;
determining, via the computing device, based on said analysis, a set of amino acid indices;
training, via the computing device, an ensemble tree classifier based on said amino acid indices;
identifying, via the computing device, a set of TCR seq sample data, said TCR seq sample data set being preprocessed and clustered according to antigen-specific groups by a deep learning algorithm executed by the computing device, said TCR seq sample data set;
applying, via the computing device, said trained tree classifier to said TCR seq sample data set; and
determining, via the computing device, based on said application, a cancer score, said cancer score providing an indication of probability of an immune repertoire being cancerous.

2. The method of claim 1, further comprising:
identifying, over a network, human reference genome information;
analyzing the human reference genome information; and
extracting, based on said analysis of the human reference genome information, CDR3 sequences.

3. The method of claim 2, further comprising:
performing, via the computing device, a pairwise alignment of the CDR3 sequences, wherein said cancer score is based on said pairwise alignment.

4. The method of claim 3, further comprising:
generating a connectivity matrix of CDR3 sequences based on said pairwise alignment, wherein said clustering is based on said generated matrix, wherein said TCRs are grouped into antigen-specific clusters, wherein said cancer score determination is based on said antigen-specific clusters.

5. The method of claim 2, wherein said extraction is performed by the computing device executing the algorithm for calling the TCR transcript hypervariable complementary determining region 3 (CDR3 regions) during said analysis.

6. The method of claim 2, further comprising:
determining, based on said computing device executing the algorithm for calling the TCR transcript hypervariable complementary determining region 3 (CDR3 regions), information indicating cancerous CDR3s and non-cancerous CDR3s from said set of amino acid indices.

7. The method of claim 1, wherein said training of the ensemble tree classifier comprises minimizing training cycles and minimizing cross-validation (CV) errors.

8. The method of claim 7, wherein said CV errors being calculated based on CDR3 length to an independent validation data value.

9. The method of claim 7, wherein said minimization of said CV errors is based on a predetermined number of sampling rounds.

10. The method of claim 1, wherein said training comprises applying an adaptive boosting algorithm.

11. The method of claim 1, wherein said training comprises applying a deep neural network algorithm.

12. A non-transitory computer-readable storage medium tangibly encoded with computer-executable instructions, that when executed by a processor associated with a computing device, performs a method comprising the steps of:
identifying, via the computing device, a set of ribonucleic acid sequence (RNA-seq) data;
identifying, via the computing device, data associated with a set of antigen-specific T cell receptors (TCRs);
analyzing, via the computing device executing an algorithm for calling a TCR transcript hypervariable complementary determining region 3 (CDR3 regions), said RNA-seq data and said TCR data;
determining, via the computing device, based on said analysis, a set of amino acid indices;
training, via the computing device, an ensemble tree classifier based on said amino acid indices;
identifying, via the computing device, a set of TCR seq sample data, said TCR seq sample data set being preprocessed and clustered according to antigen-specific groups by a deep learning algorithm executed by the computing device, said TCR seq sample data set;
applying, via the computing device, said trained tree classifier to said TCR seq sample data set; and
determining, via the computing device, based on said application, a cancer score, said cancer score providing an indication of probability of an immune repertoire being cancerous.

13. The non-transitory computer-readable storage medium of claim 12, further comprising:
identifying, over a network, human reference genome information;
analyzing the human reference genome information; and
extracting, based on said analysis of the human reference genome information, CDR3 sequences.

14. The non-transitory computer-readable storage medium of claim 13, further comprising:
performing, via the computing device, a pairwise alignment of the CDR3 sequences, wherein said cancer score is based on said pairwise alignment.

15. The non-transitory computer-readable storage medium of claim 14, further comprising:
generating a connectivity matrix of CDR3 sequences based on said pairwise alignment, wherein said clustering is based on said generated matrix, wherein said TCRs are grouped into antigen-specific clusters, wherein said cancer score determination is based on said antigen-specific clusters.

16. The non-transitory computer-readable storage medium of claim 13, wherein said extraction is performed by the computing device executing the algorithm for calling the TCR transcript hypervariable complementary determining region 3 (CDR3 regions) during said analysis.

17. The non-transitory computer-readable storage medium of claim 13, further comprising:
determining, based on said computing device executing the algorithm for calling the TCR transcript hypervariable complementary determining region 3 (CDR3 regions), information indicating cancerous CDR3s and non-cancerous CDR3s from said set of amino acid indices.

18. The non-transitory computer-readable storage medium of claim 12, wherein said training of the ensemble tree classifier comprises minimizing training cycles and minimizing cross-validation (CV) errors, wherein said CV errors being calculated based on CDR3 length to an independent validation data value, wherein said minimization of said CV errors is based on a predetermined number of sampling rounds.

19. A computing device comprising:
a processor; and
a non-transitory computer-readable storage medium for tangibly storing thereon program logic for execution by the processor, the program logic comprising:
logic executed by the processor for identifying, via the computing device, a set of ribonucleic acid sequence (RNA-seq) data;
logic executed by the processor for identifying, via the computing device, data associated with a set of antigen-specific T cell receptors (TCRs);
logic executed by the processor for analyzing, via the computing device executing an algorithm for calling a TCR transcript hypervariable complementary determining region 3 (CDR3 regions), said RNA-seq data and said TCR data;
logic executed by the processor for determining, via the computing device, based on said analysis, a set of amino acid indices;
logic executed by the processor for training, via the computing device, an ensemble tree classifier based on said amino acid indices;
logic executed by the processor for identifying, via the computing device, a set of TCR seq sample data, said TCR seq sample data set being preprocessed and clustered according to antigen-specific groups by a deep learning algorithm executed by the computing device, said TCR seq sample data set;
logic executed by the processor for applying, via the computing device, said trained tree classifier to said TCR seq sample data set; and
logic executed by the processor for determining, via the computing device, based on said application, a cancer score, said cancer score providing an indication of probability of an immune repertoire being cancerous.

20. The computing device of claim 19, further comprising:
logic executed by the processor for identifying, over a network, human reference genome information;
logic executed by the processor for analyzing the human reference genome information;
logic executed by the processor for extracting, based on said analysis of the human reference genome information, CDR3 sequences;
logic executed by the processor for performing a pairwise alignment of the CDR3 sequences, wherein said cancer score is based on said pairwise alignment; and
logic executed by the processor for generating a connectivity matrix of CDR3 sequences based on said pairwise alignment, wherein said clustering is based on said generated matrix, wherein said TCRs are grouped into antigen-specific clusters, wherein said cancer score determination is based on said antigen-specific clusters.
